(19) United States
(12) Patent Application Publication
(10) Pub. No.: US 2024/0358296 A1
(43) Pub. Date: Oct. 31, 2024

(54) Instrument Delivery Device with Integrated Scrubbing Cap and Method of Use Thereof

(71) Applicant: Becton, Dickinson and Company, Franklin Lakes, NJ (US)

(72) Inventors: Praveen Nalawade, Bangaluru (IN); Manish Kumar, Bengaluru (IN); Karthik MR, Bangalore (IN); Shishir Prasad, Ramsey, NJ (US)

(21) Appl. No.: 18/643,525

(22) Filed: Apr. 23, 2024

Related U.S. Application Data
(60) Provisional application No. 63/461,695, filed on Apr. 25, 2023.

(51) Int. Cl. A61B 5/15

(52) U.S. Cl. CPC A61B 5/150328; A61B 5/15003; A61B 5/150274

(57) ABSTRACT

Provided herein is an instrument delivery device including an instrument, a housing having a proximal end and a distal end and defining an inner volume configured to movably receive the instrument, a coupling device positioned at the distal end of the housing and including a lock configured to couple the housing to a proximal connector of the catheter assembly, and an advancement member configured to move relative to the housing to move the instrument between a first position and a second position. A scrubbing cap is engaged with the lock that includes a scrubbing insert, a housing having a connector portion configured to engage with the lock, a holder portion configured to retain the scrubbing insert therein, and a seal that seals the scrubbing insert within the holder portion. The scrubbing cap is engaged with the lock so as to be removable from the instrument delivery device.

### CROSS-REFERENCE TO RELATED APPLICATION

The present application claims priority to U.S. Provisional Application No. 63/461,695 entitled “Instrument Delivery Device with Integrated Scrubbing Cap and Method of Use Thereof” filed Apr. 25, 2023, the entire disclosure of which is incorporated by reference.

### BACKGROUND OF THE INVENTION

### Field of the Invention

The present disclosure relates to a catheter system with an integrated scrubbing cap.

### Description of Related Art

Catheters are commonly used to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

In many VADs, when utilizing an IV catheter, a needleless connector is used to close off the VAD (i.e., to seal off a hub or port of the VAD), with the needleless connector then subsequently accessed to introduce an instrument into the patient (via the catheter) to draw blood, administer medication or other necessary fluids, and/or provide for blood-related monitoring or analysis. That is, a blood draw device or other instrument delivery device may couple with the needleless connector to provide for introduction and advancement of a catheter or other instrument (e.g., guidewire, probe, etc.) into the IV catheter. In such arrangements, it is essential that the needleless connector be properly cleaned and sterilized prior to engagement of the device therewith to prevent microbial ingress and possible catheter-related blood stream infections (CRBSIs). Currently, clinical practitioners often use alcohol wipes for scrubbing and disinfecting the needleless connector before connecting a blood draw or instrument delivery device. However, use of alcohol wipes for scrubbing and disinfecting the needleless connector in this manner is oftentimes not completely effective in disinfecting all internal/external surfaces of the needleless connector, including the intricate thread areas around the luer portion thereof that couples with the blood draw or instrument delivery device. Additionally, use of alcohol wipes for scrubbing and disinfecting the needleless connector does not comply to aseptic non-touch technique (ANTT) standards.

Accordingly, a need exists in the art for a scrubbing device that can disinfect a needleless connector (or other access port) of a peripheral IV catheter before connecting of a blood draw or other instrument delivery device thereto, with such a scrubbing device meeting ANTT standards.

### SUMMARY OF THE INVENTION

Provided herein is an instrument delivery device coupleable to a catheter assembly including a catheter positionable intravenously in a patient. The instrument delivery device includes an instrument configured to be inserted within the catheter, a housing having a proximal end and a distal end and defining an inner volume configured to movably receive the instrument, a coupling device positioned at the distal end of the housing and including a lock configured to couple the housing to a proximal connector of the catheter assembly, and an advancement member configured to move relative to the housing to move the instrument between a first position, in which a distal end of the instrument is disposed within the housing or within the lock, and a second position, in which the distal end of the instrument is disposed within the catheter or past a distal tip of the catheter. The instrument delivery device also includes a scrubbing cap engaged with the lock, with the scrubbing cap further including a scrubbing insert, a housing including a connector portion configured to engage with the lock to secure the scrubbing cap to the instrument delivery device and a holder portion distal from the connector portion and including a cavity configured to retain the scrubbing insert therein, and a seal attached to the holder portion to seal the scrubbing insert within the cavity. The scrubbing cap is engaged with the lock in a manner that provides for removal of the scrubbing cap from the instrument delivery device.

In some embodiments, the connector portion comprises a protrusion having a distal end joined with the connector portion and a proximal end having an opening formed therein, the opening providing access to a channel formed within the protrusion.

In some embodiments, the lock includes an elongated member insertable within the channel of the protrusion and a pair of locking arms positioned on opposing sides of the elongated member and enagageable with the protrusion.

In some embodiments, the protrusion of the connector portion includes a first portion having a first outer diameter, a second portion positioned proximally from the first portion and having a second outer diameter less than the first outer diameter, the second portion including the opening, and an annular flange disposed between the first portion and the second portion and forming a lip, wherein the annular flange is positioned such that a hooked end portion of each of the pair of locking arms engages the lip of the annular flange when the elongated member is inserted into the channel.

In some embodiments, the first portion includes a pair of retaining stops formed on an outer surface thereof and adjacent the annular flange, the pair of retaining stops arranged to form a retaining seat that receives the hooked end portion of a respective locking arm of the pair of locking arms, with the retaining seat preventing circumferential displacement of the locking arm.

In some embodiments, the channel includes a first channel portion formed in the first portion of the protrusion and a second channel portion formed in the second portion of the protrusion, wherein the first channel portion has a first channel diameter and the second channel portion has a second channel diameter greater than the first channel diameter.

In some embodiments, the elongated member includes a male luer portion and a blunted cannula portion extending out distally from the male luer portion, with the blunted cannula portion sized to fit in the first channel portion and the male luer portion sized to fit in the second channel portion.

In some embodiments, the elongated member is a male luer connection sized to fit in the second channel portion, and the first channel portion remains open when the elongated member is inserted into the channel.

In some embodiments, the lock includes an elongated member insertable within the channel of the protrusion and a collar positioned about the elongated member and including a threaded inner surface.

In some embodiments, the protrusion of the connector portion comprises a threaded outer surface configured to engage with the threaded inner surface of the collar to secure the scrubbing cap to the lock.

In some embodiments, the elongated member is a male luer connection sized to fit in the channel.

In some embodiments, the protrusion of the connector portion includes a first portion and a second portion positioned proximally from the first portion, wherein the second portion includes the opening and the threaded outer surface is formed on the second portion.

In some embodiments, the channel includes a first channel portion formed in the first portion of the protrusion and a second channel portion formed in the second portion of the protrusion, wherein the first channel portion has a first channel diameter and the second channel portion has a second channel diameter greater than the first channel diameter.

In some embodiments, the elongated member includes a male luer portion and a blunted cannula portion extending out distally from the male luer portion, with the blunted cannula portion sized to fit in the first channel portion and the male luer portion sized to fit in the second channel portion.

In some embodiments, the cavity in the holder portion and the scrubbing insert are sized and reconfigured to receive a female luer connection of the proximal connector of the catheter assembly.

Also provided herein is a method of using an instrument delivery device having a scrubbing cap integrated therewith. The method includes removing the seal from the holder portion, positioning the proximal connector of the catheter assembly into the holder portion, such that the proximal connector contacts the scrubbing insert, and cleaning the proximal connector with the scrubbing insert via application of a twisting motion to the instrument delivery device.

In some embodiments, the method includes detaching the scrubbing cap from the lock subsequent to cleaning of the proximal connector and coupling the lock to the proximal connector, wherein, upon coupling of the lock to the proximal connector, the instrument delivery device is operable to move the instrument between from the first position to the second position.

Also provided herein is a method of manufacturing an instrument delivery device coupleable to a catheter assembly including a catheter positionable intravenously in a patient. The method includes providing an instrument delivery device including an instrument configured to be inserted within the catheter, a housing having a proximal end and a distal end and defining an inner volume configured to movably receive the instrument, a coupling device positioned at the distal end of the housing and including a lock configured to couple the housing to a proximal connector of the catheter assembly, and an advancement member configured to move relative to the housing to move the instrument between a first position, in which a distal end of the instrument is disposed within the housing or within the lock, and a second position, in which the distal end of the instrument is disposed within the catheter or past a distal tip of the catheter. The method also includes engaging a scrubbing cap with the lock, the scrubbing cap including a scrubbing insert, a housing including a connector portion configured to engage with the lock to secure the scrubbing cap to the instrument delivery device and a holder portion distal from the connector portion and including a cavity configured to retain the scrubbing insert therein, and a seal attached to the holder portion to seal the scrubbing insert within the cavity. The scrubbing cap is engaged with the lock in a manner that provides for removal of the scrubbing cap from the instrument delivery device.

In some embodiments, engaging the scrubbing cap with the lock includes inserting an elongated member of the lock into a channel formed in the connector portion of the scrubbing cap and pinching a pair of locking arms of the lock onto an annular flange formed about a protrusion of the connector portion.

In some embodiments, a locking arm of the pair of locking arms is secured between a pair of retaining stops formed on the protrusion of the connector portion, thereby preventing circumferential displacement of the pair of locking arms relative to the connector portion.

In some embodiments, engaging the scrubbing cap with the lock includes inserting an elongated member of the lock into a channel formed in the connector portion of the scrubbing cap and threadingly engaging an internally threaded collar of the lock with a threaded outer surface of a protrusion of the connector portion.

### BRIEF DESCRIPTION OF THE DRAWINGS

FIG. 1 is a perspective view of a catheter system, according to an aspect or embodiment of the present application;

FIG. 2 is a perspective view of an instrument delivery device of the system of FIG. 1, according to an aspect or embodiment of the present application;

FIG. 3 is an exploded view of the instrument delivery device of FIG. 2;

FIG. 4 is a side cross-sectional view of the instrument delivery device of FIG. 2, showing an instrument in a first, retracted position;

FIG. 5 is a side cross-sectional view of the instrument delivery device of FIG. 2, showing the instrument in a second, extended position;

FIG. 6A is an exploded view of a scrubbing cap included in the instrument delivery device of FIG. 2, along with a coupling device of the instrument delivery device, according to an aspect or embodiment of the present application;

FIG. 6B is a detailed view of the scrubbing cap of FIG. 6A mated with the coupling device;

FIG. 7 is a cross-sectional view of the scrubbing cap and coupling device of FIG. 6A;

FIG. 8 is a cross-sectional view of the scrubbing cap and coupling device of FIG. 6A, with the scrubbing cap positioned over a proximal coupler;

FIG. 9 is an exploded view of a scrubbing cap and associated coupling device useable with the system of FIG. 1, according to an aspect or embodiment of the present application;

FIG. 10 is a cross-sectional view of the scrubbing cap and coupling device of FIG. 9, with the scrubbing cap positioned over a proximal coupler;

FIG. 11 is an exploded view of a scrubbing cap and associated coupling device useable with the system of FIG. 1, according to an aspect or embodiment of the present application;

FIG. 12 is a cross-sectional view of the scrubbing cap and coupling device of FIG. 11, with the scrubbing cap positioned over a proximal coupler;

FIG. 13 is an exploded view of a scrubbing cap and associated coupling device useable with the system of FIG. 1, according to an aspect or embodiment of the present application;

FIG. 14 is a cross-sectional view of the scrubbing cap and coupling device of FIG. 13, with the scrubbing cap positioned over a proximal coupler;

FIG. 15 is an exploded view of a scrubbing cap and associated coupling device useable with the system of FIG. 1, according to an aspect or embodiment of the present application;

FIG. 16 is a cross-sectional view of the scrubbing cap and coupling device of FIG. 15, with the scrubbing cap positioned over a proximal coupler;

FIG. 17 is an exploded view of a scrubbing cap and associated coupling device useable with the system of FIG. 1, according to an aspect or embodiment of the present application;

FIG. 18 is a cross-sectional view of the scrubbing cap and coupling device of FIG. 17, with the scrubbing cap positioned over a proximal coupler;

FIG. 19 is an exploded view of a scrubbing cap and associated coupling device useable with the system of FIG. 1, according to an aspect or embodiment of the present application; and

FIG. 20 is a cross-sectional view of the scrubbing cap and coupling device of FIG. 19, with the scrubbing cap positioned over a proximal coupler.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

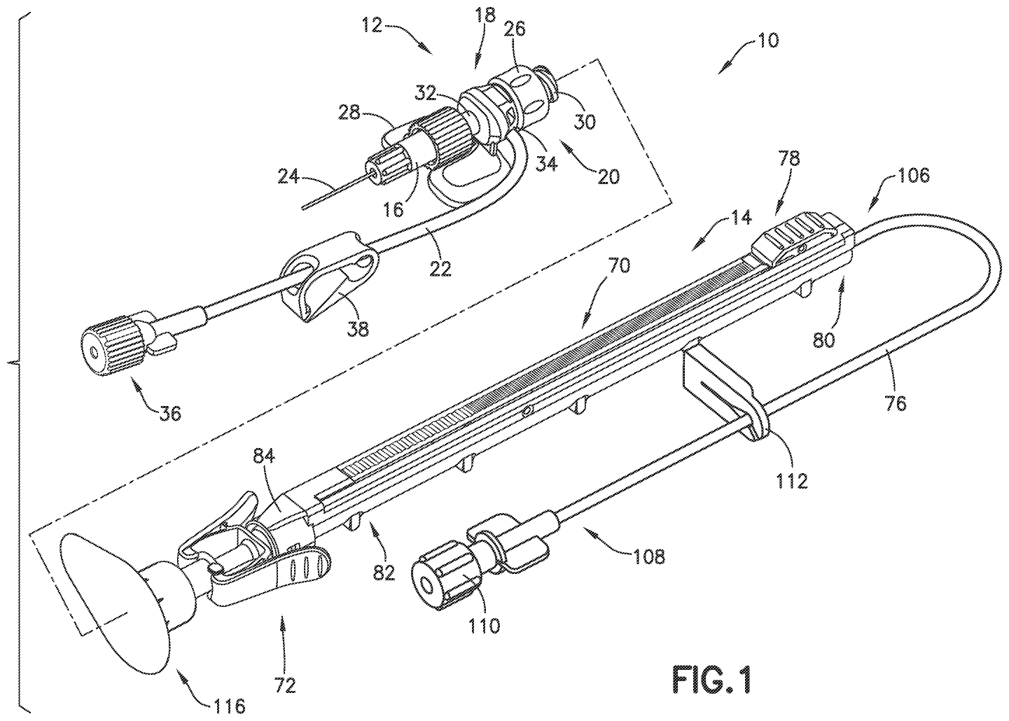

Referring to FIG. 1, shown is a non-limiting embodiment of a catheter system 10, according to one aspect or embodiment with which embodiments of the invention may be implemented. The catheter system 10 includes a catheter assembly 12 with which an associated instrument delivery device 14 may be used. While the instrument delivery device 14 is described here below as a delivery device that introduces a catheter tube into the catheter assembly 12 for performing a blood draw (i.e., a “blood draw device”), it is recognized that the instrument delivery device could instead deliver another suitable instrument into the vasculature of a patient, such as a guide wire or sensing probe, as other non-limiting examples.

In accordance with one embodiment, the catheter assembly 12 includes a catheter hub 16 and a non-integrated extension set 18 that includes a catheter stabilizing catheter connector 20 (hereafter “catheter connector 20”), along with extension tubing 22. The catheter hub 16 receives a catheter 24 and is coupled to the catheter connector 20. In one aspect or embodiment, the catheter hub 16 may be an AccuCath™ catheter system commercially available from Becton, Dickinson and Company.

The catheter connector 20 is configured to be placed in contact with the skin surface of a patient at or near an insertion site of catheter 24. The catheter connector 20 can be any suitable shape, size, and/or configuration. In the illustrated non-limiting embodiment, the catheter connector 20 has a connector portion 26 and a stabilization portion 28. The connector portion 26 has a proximal port with a proximal coupler 30, a distal port with a distal coupler 32, and defines at least one lumen (not shown) extending through or otherwise in fluid communication with the couplers 30, 32. The proximal coupler 30 and/or the distal coupler 32 can be, for example, male or female luer locks and/or any other suitable coupler. In the illustrated embodiment, the proximal coupler 30 is provided as a needleless access connector (i.e., needle-free connector (NFC))—hereafter “needleless access connector 30”—that can be physically and fluidically coupled to peripheral devices such as, e.g., a blood draw device (e.g., PIVO™ from Becton, Dickinson and Company), or a vascular access probe (VAP) for in-vein digital measurement of patient data such as temperature, pH, lactate, and/or other blood-based measurements. The proximal coupler 30 presents a female luer connection/lock to which such peripheral devices may be connected. The distal coupler 32 can be physically and fluidically coupled to catheter hub 16 such that the lumen of the connector portion 26 is at least selectively in fluid communication with the catheter 24. In some embodiments, an inner surface of the connector portion 26 can be configured to provide alignment, guidance, centering, etc., to an object or device (e.g., a blood draw catheter or the like, as described above) being advanced therethrough.

The connector portion 26 may also include and/or define one or more additional ports, such as a side port 34. Side port 34 can be included in and/or can be a part of the connector portion 26, the proximal coupler 30, the distal coupler 32, and/or a combination thereof. In some instances, positioning side port 34 in a desired position along a length of the connector portion 26, between the couplers 30, 32 can allow for a reduced length of the connector portion 26 and/or can facilitate flushing and/or fluid transfer via side port 34.

Side port 34 defines a lumen (not shown) that is in fluid communication with the lumen between distal coupler 32 and proximal coupler 30. In other words, the connector portion 26 and/or the side port 34 can include and/or define a first lumen (e.g., between distal coupler 32 and proximal coupler 30) and a second lumen (e.g., lumen defined by side port 34 and extension tubing 22). As such, side port 34 can provide access to the lumen between distal coupler 32 and proximal coupler 30, which in turn can provide access to a device (e.g., a VAD) that is coupled to the distal coupler 32 and/or can provide access to a portion of the body in which the VAD is at least partially disposed.

In some embodiments, the arrangement of side port 34 can be such that the connector portion 26 forms, for example, a Y-connector or a T-connector. More particularly, side port 34 can be disposed substantially perpendicular (e.g., about 90 degrees) to the lumen of the connector portion 26 and near or adjacent the proximal coupler 30. In non-limiting embodiments, side port 34 extends from connector portion 26 at an angle, relative to the lumen between distal coupler 32 and proximal coupler 30, that is not 90 degrees (e.g., side port 34 extends at an angle of, for example and without limitation, 15-165 degrees, with all values and subranges therebetween inclusive). In non-limiting embodiments, more than one side port 34 is included in connector portion 26.

As shown in FIG. 1, side port 34 may be coupled to extension tubing 22 such that the extension tubing 22 is in fluid communication with the lumen of the side port 34. In some embodiments, the side port 34 and/or the extension tubing 22 can be and/or can form at least a portion of a fluid line that can be used to deliver fluid, remove fluid, flush fluid, and/or the like. In such embodiments, for example, an arrangement in which side port 34 is disposed adjacent to the proximal coupler 30 can enable flushing of the proximal coupler 30, any valve(s) included therein, and/or a space between any valve(s) and an inner surface of the connector portion 26 (e.g., defining at least a portion of the lumen).

As shown, the extension set 18 includes a proximal access port 36 coupled to a proximal end portion of the extension tubing 22, with the extension tubing 22 in fluid communication with the side port 34 of the catheter connector 20. A clamp 38 may be provided on the extension tubing 22, with the clamp 38 configured to selectively restrict flow through the extension tubing 22.

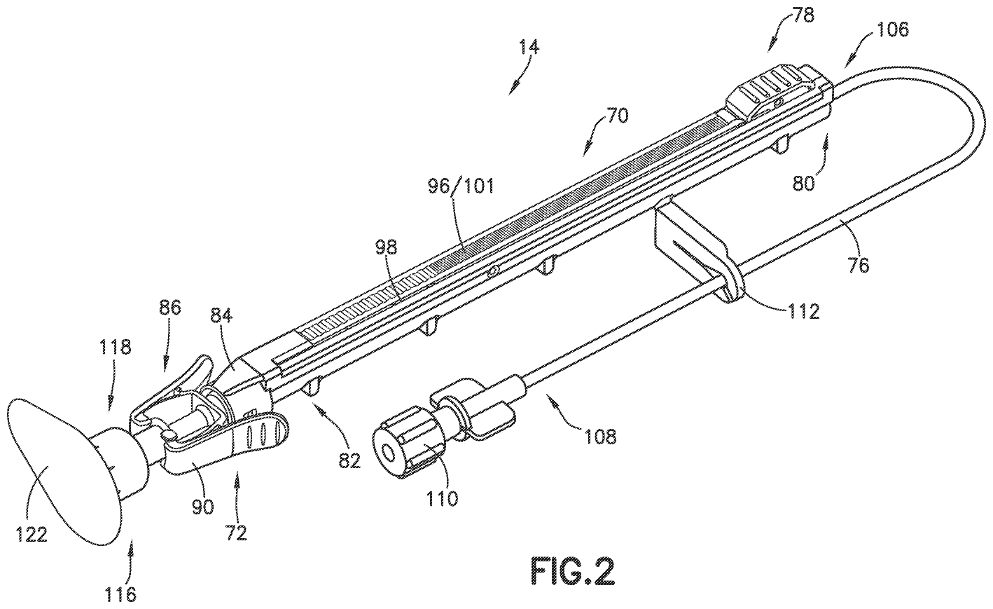

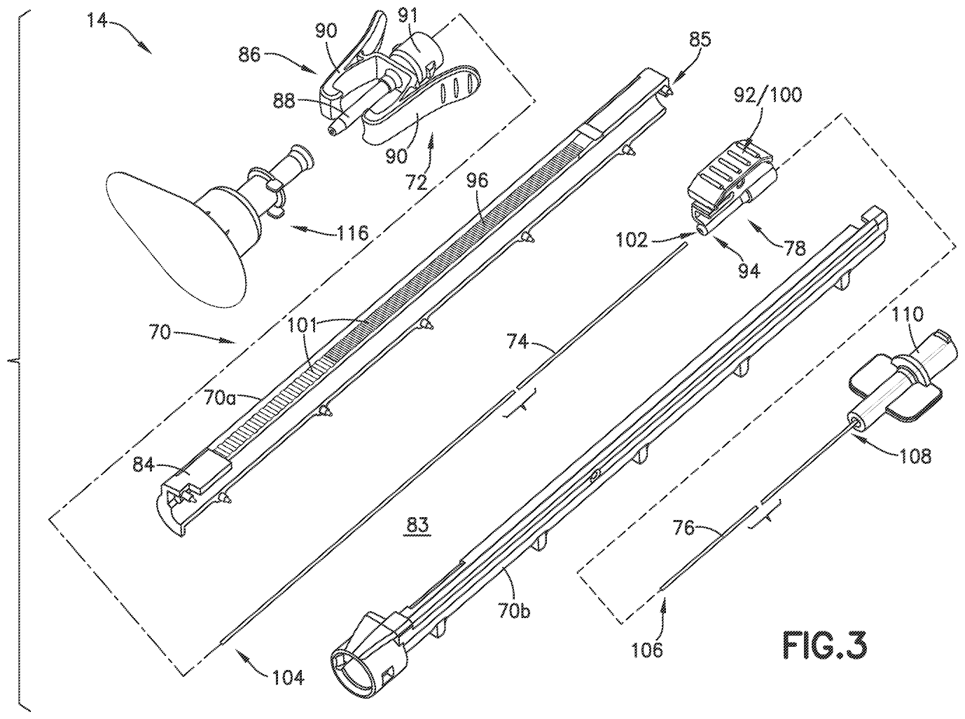

Referring still to FIG. 1 and now also to FIGS. 2 and 3, the instrument delivery device 14 (hereafter “blood draw device 14”) includes a housing 70, a coupling device 72, a catheter 74, a secondary catheter 76, and an advancement member 78. As described in further detail herein, a user can transition the blood draw device 14 from a first configuration to a second configuration to advance the catheter 74 through an existing, placed, and/or indwelling catheter 24 (i.e., when the blood draw device 14 is coupled thereto) such that at least an end portion of the catheter 74 is disposed in a distal position relative to the indwelling catheter 24. Moreover, with indwelling catheters each having a shape, size, and/or configuration that can vary based on, for example, a manufacturer of the indwelling catheter 24 and/or its intended usage, the blood draw device 14 can be arranged to be coupled to an indwelling catheter 24 having any suitable configuration and subsequently, to advance at least a portion of a catheter through the indwelling catheter 24. In addition, the blood draw device 14 can be manipulated by a user to place a distal surface of the catheter 74 a predetermined and/or desired distance beyond a distal surface of the indwelling catheter 24 to be disposed within a portion of a vein that receives a substantially unobstructed flow of blood.

The housing 70 of blood draw device 14 can be an elongate member having a proximal end 80 and a distal end 82 and defining an inner volume 83. In some embodiments, the housing 70 may be formed of a pair of housing portions 70a, 70b that are coupled together to define the inner volume 83. The housing 70 may include one or more features or surface finishes on an outer surface thereof that can be arranged to increase the ergonomics of the blood draw device 14, which in some instances can allow a user to manipulate the blood draw device 14 with one hand (i.e., single-handed use). The distal end 82 of the housing 70 can include a coupler 84 formed thereon configured to mate with the coupling device 72 of blood draw device and, in one embodiment, the coupler 84 may include a threaded inner surface that engages the coupling device 72. Additionally, the proximal end 80 of the housing 70 can include an opening or port 85 configured to receive a portion of the secondary catheter 76 (i.e., the portion of the secondary catheter 76 extending proximally out from housing 70) and provide for advancement and retraction of the secondary catheter 76 through the port 85.

The coupling device 72 of blood draw device 14 is provided at the distal end 82 of the housing 70, with the coupling device 72 providing for reversible coupling of the blood draw device 14 to catheter assembly 12, such as via proximal coupler 30 as shown in FIG. 1. As previously described, proximal coupler 30 may be provided as a needleless access connector, and is thus referred to hereafter as “needleless access connector 30.” In some embodiments, the coupling device 72 is configured as a lock 86 that includes a blunted cannula 88 and locking arms 90 for coupling to the needleless access connector 30 of catheter assembly 12, with the blunted cannula 88 and locking arms 90 forming three points of contact therewith. However, those of skill will appreciate that any connection or coupling, for example a luer, can be used, so long as a distal end of catheter 74 may pass through the coupling device 72 to access catheter assembly 12. Additionally, a proximal end of the coupling device 72 may be configured as a threaded connection 91 (i.e., a threaded outer surface) that is configured to engage the coupler 84 of housing 70—with the threaded connection 91 of coupling device 72 engaging the threaded inner surface of coupler 84.

In accordance with some aspects of the disclosure, the advancement member 78 of blood draw device 14 includes a first portion 92 and a second portion 94. The first portion 92 is movably disposed along an upper surface 96 of the housing 70 and the second portion 94 is movably disposed within the inner volume 83 of the housing 70. The arrangement of the advancement member 78 and the housing 70 is such that a connecting portion (not shown) of the advancement member 78 that joins the first and second portions 92, 94 is seated within a slot 98 formed in the upper surface 96 of the housing 70—the slot 98 generally extending between the proximal and distal ends 80, 82 of the housing 70. As the first and second portions 92, 94 are joined together, movement of the first portion 92 along the upper surface 96 of the housing 70 results in a corresponding movement of the second portion 94 within the inner volume 83.

As shown in FIGS. 2 and 3, the first portion 92 of the advancement member 78 may be configured as a tab 100 having a contact surface engageable by a user and an underside that is in contact with the outer surface 96 of the housing 70. In such embodiments, the upper surface 96 of the housing 70 can include a track 101, for example, a set of ribs, ridges, bumps, grooves, and/or the like along which the underside of tab or protrusion advances when the advancement member 78 is engaged by a user. In this manner, a user can engage the first portion 92 of the advancement member 78 and can move the advancement member 78 relative to the housing 70.

As further shown in FIG. 3, the second portion 94 includes an opening 102 extending therethrough that is configured to grip or retain a portion of the catheter 74. Due to a portion of the catheter 74 being retained within the opening 102 of second portion 94, movement of the advancement member 78 relative to housing 70 causes a corresponding movement of the catheter 74 relative to the housing 70. In this manner, a distal end 104 of the catheter 74 can be selectively moved out of or back into the inner volume 83 of the housing 70 as desired, such as advancing the distal end 104 of the catheter 74 out of the housing 70 when the blood draw device 14 has been coupled to the catheter assembly 12 and a blood draw procedure is to be performed.

In addition to receiving catheter 74 therein, the second portion 94 also grips or retains a portion of secondary catheter 76 therein. That is a distal end 106 of secondary catheter 76 is secured to second portion 94 (i.e., in opening 102 thereof) and extends proximally out therefrom, with the secondary catheter 76 extending out through opening 85 of the housing 70. A proximal end 108 of the secondary catheter 76 may include a coupler 110 thereon that may be coupled to a fluid reservoir, fluid source, syringe, evacuated container, or pump, with the secondary catheter 76 establishing fluid communication between the reservoir, source, pump, etc. and the catheter 74. Additionally, in some embodiments, the secondary catheter 76 may also have a clamp 112 provided thereon, such as a slide clamp or pinch clamp, to facilitate stoppage of flow through the secondary catheter 76.

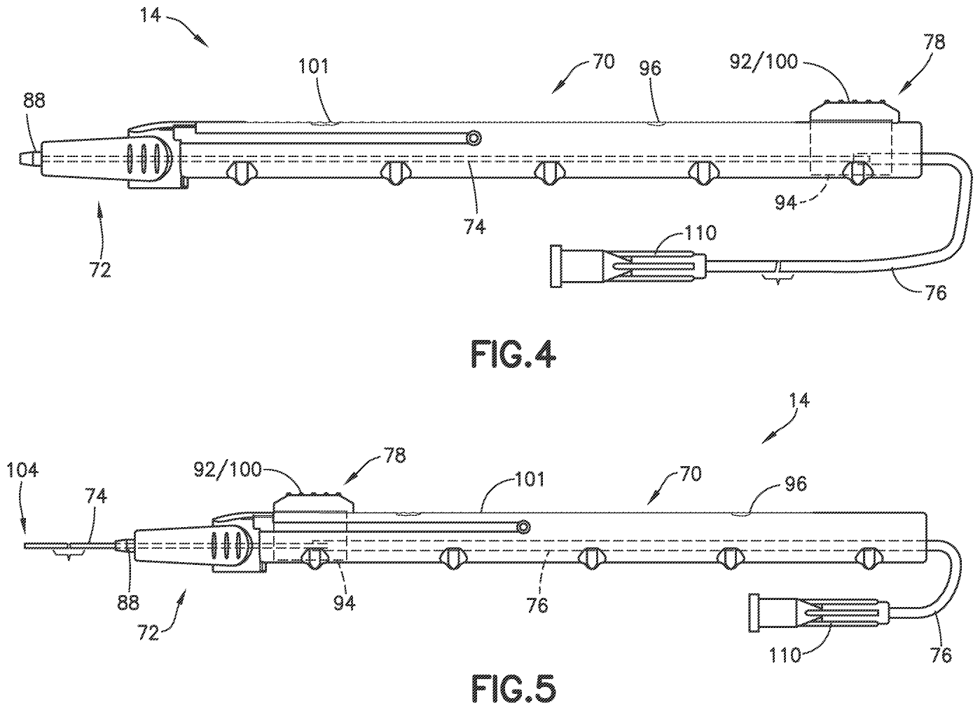

Referring now to FIGS. 4 and 5, changing of the blood draw device 14 between the first configuration and second configuration is shown. The blood draw device 14 can be in the first configuration prior to use and can be transitioned by a user (e.g., a doctor, physician, nurse, technician, phlebotomist, and/or the like) from the first configuration (FIG. 4) to the second configuration (FIG. 5) to dispose at least the distal end 104 of the catheter 74 in a distal position relative to the housing 70.

The blood draw device 14 is in the first configuration of FIG. 4 when the catheter 74 is disposed in the first position within the housing 70. In some embodiments, substantially the entire catheter 74 is disposed within the housing 70 when the catheter 74 is in the first position. In other embodiments, the catheter 74 is disposed within the housing 70 and the lock 86 when catheter 74 is in the first position.

The advancement member 78 is disposed in a proximal position when the blood draw device 14 is in the first configuration, and the user may engage the tab 100 of advancement member 78 to move the advancement member 78 relative to the housing 70, which in turn, moves the catheter 74 from the first position (e.g., disposed within the housing 70) toward the second position. In this manner, the catheter 74 is moved through the inner volume 83 of housing 70 and through the lock 86 and, as such, at least the distal end 104 of the catheter 74 is disposed outside of and distal to the lock 86.

The blood draw device 14 is in the second configuration of FIG. 5 when the catheter 74 is disposed in the second position. The second position of the catheter 74 is reached when the distal end 104 of the catheter 74 is placed in a desired position relative to a distal end of the catheter 24. In some instances, for example, a distal end 104 of the catheter 74 can be substantially flush with a distal end of the catheter 24 when the catheter 74 is in the second position. In other instances, the distal end 104 of the catheter 74 can extend a predetermined distance beyond the distal end of the catheter 24, such that the distal end 104 of the catheter 74 is positioned within the vein at a predetermined distance beyond the distal end of the catheter 74 (e.g., a position within a vein that is substantially free from debris (e.g., fibrin/blood clots) otherwise surrounding the distal end portion of the catheter 24).

With the catheter 74 in the second position (e.g., with the blood draw device 14 in the second configuration shown, for example, in FIG. 5), the user can establish fluid communication between a fluid reservoir, fluid source, syringe, and/or the like and the catheter 74. The blood draw device 14 can then aspirate a volume of blood from the vein based at least in part on disposing the distal end 104 of the catheter 74 at the predetermined and/or desired distance beyond the distal surface of the catheter 24 (FIG. 1). In some instances, once a desired amount of blood has been collected and/or once a desired volume of a drug has been delivered to the patient, the user can move the advancement member 78 in the proximal direction, such as moving the advancement member 78 back to its proximal most position, so as to move the catheter 74 back to the first position.

While instrument delivery device 14 is shown and described in FIGS. 1-5 as having an advancement member 68 having first and second portions 92, 94 configured formed to move along an upper surface 96 of housing 70 and within the inner volume 83 thereof, it is recognized that instrument delivery device 14 may have an advancement member of alternate configurations. For example, instrument delivery device 14 may be configured to have a nested housing arrangement—where the advancement member is provided as a telescopic cylinder that may move within housing 70 to advance (and retract) catheter 74, according to another embodiment. It is recognized that still other advancement member configurations may be implemented with instrument delivery device 14, according to aspects of the disclosure.

It is recognized that when initially connecting the blood draw device 14 to catheter assembly 12—via coupling of lock 86 with needleless access connector 30—it is desirable to disinfect the needleless access connector 30 prior to connection of blood draw device 14 thereto. That is, it is desirable to clean and sterilize the needleless access connector 30 prior to engagement of lock 86 therewith (e.g., via insertion of blunt cannula 88 into needleless access connector 30) to prevent microbial ingress and possible catheter-related blood stream infections (CRBSIs).

According to aspects of the disclosure, a scrubbing cap 116 is integrated in with the blood draw device 14 to provide for cleaning and sterilizing of the needleless access connector 30 prior to engagement of the blood draw device 14 therewith. The scrubbing cap 116 is secured onto lock 86 and is configured to engage with needleless access connector 30 to enable the cleaning and sterilizing of internal and/or external surfaces thereof, such as via a twisting and scrub motion between the scrubbing cap 116 and needleless access connector 30. The scrubbing cap 116 may be removed from lock 86 upon completion of the cleaning and sterilizing of the needleless access connector 30, thereby enabling a subsequent coupling of lock 86 to needleless access connector 30 and advancement of the catheter 74 of blood draw device 14 into catheter assembly 12.

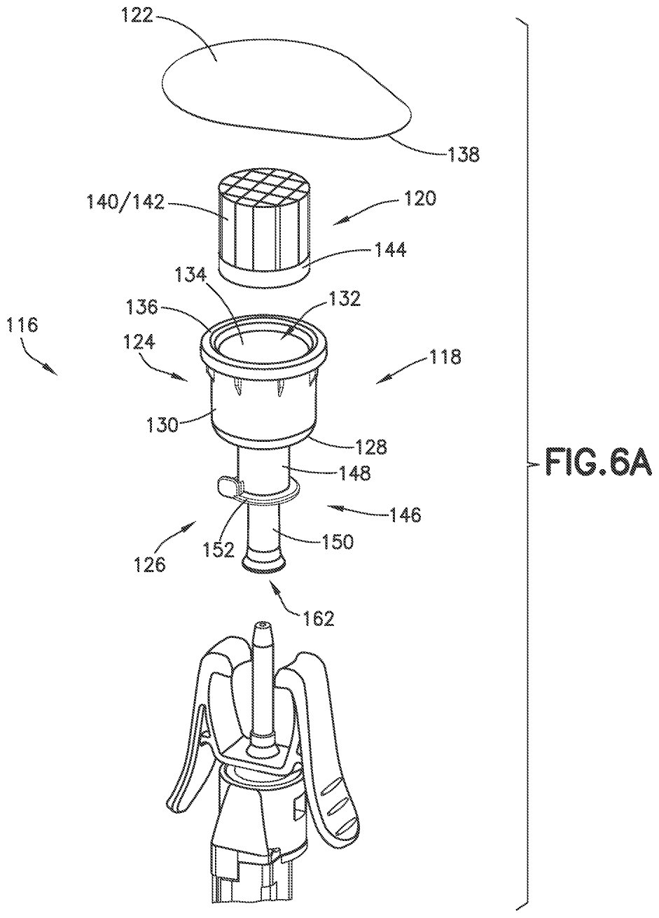

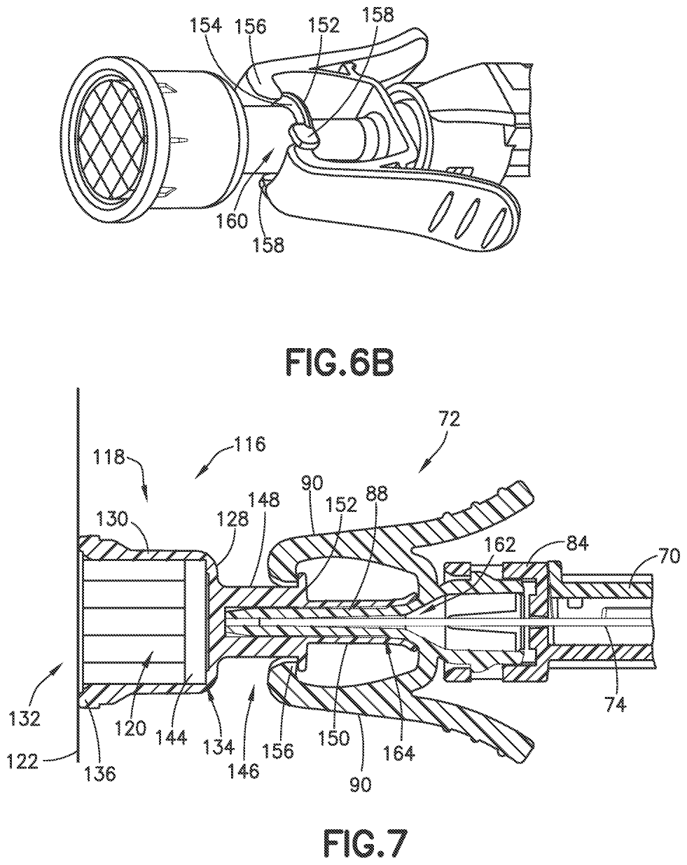

As shown in FIGS. 1-3 and now also in FIGS. 6-8, scrubbing cap 116 includes a cap housing 118, scrubbing insert 120, and a pealable seal 122. The housing 118 may be an integrally formed structure made by injection molding and may be made of an alcohol compatible material, such as polypropylene or polyethylene, for instance. The housing 118 may generally be described as including a holder portion 124 that retains the insert 120 therein and a connector portion 126 configured to mate with the coupling device 72 (i.e., lock 86) of blood draw device 14.

The holder portion 124 may have a cup shape that is formed by a base 128 and a sidewall structure 130 that defines an opening 132 on one end thereof. The sidewall structure 130 is formed integrally with the base 128 to define a cavity 134 configured to receive the insert 120 therein. In some embodiments, the cavity 134 may have a cylindrical cross-sectional shape, as shown in FIGS. 6-8, and may have a depth suitable to receive the insert 120 therein in a recessed manner. However, it is appreciated that the holder portion 124 and the cavity 134 defined thereby can assume other shapes, including square, hexagonal, etc., and that the holder portion 124, its cavity 134, and the insert 120 disposed therein can be configured in shape and size so as to enable the scrubbing cap 116 to cleanse a particular size and configuration of a medical device with which the scrubbing cap 116 is to be employed (i.e., catheter connector 20 of catheter assembly 12).

The holder portion 124 may be sized such that the cavity 134 defined by sidewall structure 130 compresses the insert 120 when the insert 120 is fitted into the cavity 134, so as to retain the insert 120 therein. A suitable hot melt glue or other suitable adhesive may also be used to adhere the insert 120 to the bottom of the holder portion 124 (i.e., to base 128), although it is recognized that other suitable methods can also be employed to secure the insert 120 to the holder portion 124, including mechanical fixation for instance. An annular lip 136 may be formed on the holder portion 124 (i.e., on sidewall structure 130) about opening 132 to define a land for receiving the seal 122, with the seal 122 cooperating with the annular lip 136 to seal the opening 132 of the cavity 134 and retain the insert 120 therein. The seal 122 seals the cavity 134 of the holder portion 124 and the insert 120 therein against contamination from the outside environment and provides a leak-proof barrier, thereby protecting the contents of insert 120 and maintaining a sealed, sterilized environment. The seal 122 provides a sufficient seal at a range of temperatures, pressures, and humidity levels and, according to embodiments, may be formed as an aluminum or multi-layer polymer film peel back top. In some embodiments, the seal 122 is heat-scaled or induction scaled to the open end of the scrubbing cap 116. The seal 122 can include a tab 138 to facilitate the manipulation of and removal of the seal 122 from the scrubbing cap 116.

The insert 120 is constructed of a foam material, for example, of injection molded construction or the insert 120 may be die-cut from a foam sheet. The foam insert 120 may be open celled, semi-opened or closed celled, according to embodiments. The insert 120 includes a cleansing substance impregnated therein (while in the holder portion 124), such as a solution of a suitable microbiocide or germicide. The cleansing substance can include an anti-bacterial disinfectant of any suitable type and suitable amount depending upon the size of the insert of foam material. For example, in one embodiment use is made of an aqueous solution including about two percent (2%) chlorhexidine gluconate (chlorhexidine solution, “CHG”) by volume in an amount of from about 0.20 cc to about 0.75 cc. Optionally, a solution including about 0.50 cc is employed. In another embodiment, a solution including about 70 percent (70%) isopropyl alcohol (“IPA”) in an aqueous solution is included in the cleansing substance. In yet another embodiment, a solution including about 70 percent (70%) IPA and about two percent (2%) CHG in an aqueous solution in an amount of about 0.2 ml is included in the cleansing substance. In the latter solution, it is recognized that the concentration of IPA can vary from about 60 percent (60%) to about 90 percent (90%) and the concentration of CHG can vary from about one percent (1%) to about five percent (5%), in one embodiment. Other suitable solution compositions and concentrations are also possible. For instance, povidone iodine or hydrogen peroxide solutions can be included in the cleansing substance, in one embodiment.

According to aspects of the disclosure, the insert 120 may be constructed (e.g., molded) to have a predefined shape that conforms to the unique shape of the patient end connector (i.e., proximal connector 30) that is to be cleaned thereby. The insert 120 may further have any of a number of constructions that provide for effective cleaning of the connector, such as including a patterned or roughened top surface and/or gaps or slits formed in the foam material thereof that enables the insert to better deform/conform about interior and exterior surfaces of the connector. In one embodiment, insert 120 is constructed to have a cylindrical shape, with the insert 120 being digitated, i.e., cross-cut with slits 140 in each of two perpendicular directions, to form separate fingers 142. The slits 140 extend downwardly a portion of the height of the insert 120, e.g., so that the fingers 142 extend integrally and upwardly from a common base 144 of cylindrical cross-sectional shape. It is also recognized that the insert 120 could have a cubic shape, such that the fingers 142 each have a rectangular cross-sectional shape. The insert 120, when disposed in the cavity 134 of the holder portion 124, substantially occupies a cross-sectional area of the cavity 134. This enables the fingers 142 to be disposed across a cross-sectional area sufficient to clean both exterior and interior surfaces of a portion of a connector (i.e., proximal connector 30) inserted into the holder portion 124, as described in greater detail below.

The connector portion 126 of scrubbing cap 116 extends proximally away from holder portion 124 and is configured to mate with blood draw device 14 to secure the scrubbing cap 116 thereto. That is, the connector portion 126 is configured to mate with lock 86 of blood draw device 14, such that the connector portion 126 is secured in place relative to lock 86.

The connector portion 126 is generally constructed as an elongated protrusion 146 extending outwardly from the base 128 of holder portion 124. The protrusion 146 may have a stepped profile, with a first portion 148 of the protrusion 146 positioned adjacent the base 128 and a second portion 150 of the protrusion 146 positioned proximal from the first portion 148. The first portion 148 of protrusion 146 is formed to have a first diameter that is larger than a second diameter of the second portion 150. An annular flange 152 may be formed at the transition between the first portion 148 and the second portion 150, with the annular flange 152 extending outwardly from the first portion 148 so as to provide a lip 154. The lip 154 provided by annular flange 152 may have a depth sufficient to provide for engagement of locking arms 90 of lock 86 therewith—with a hooked end portion 156 of each of locking arms 90 engaging a distal-facing surface of the lip 154. A pair of retaining stops 158 may be formed on the first portion 148 of protrusion 146 adjacent the distally-facing surface of annular flange 152. The retaining stops 158 are spaced apart to form a retaining seat 160 therebetween, with the retaining seat 160 sized to receive one of the respective locking arms 90 of lock 86 therein. With the locking arms 90 clamped onto protrusion 146 and engaging the distal-facing surface of the lip 154, and with one of locking arms 90 received in retaining seat 160, displacement of the locking arms 90 circumferentially about the connector portion 126 is prevented, such as might occur when the blood draw device 14 is rotated/twisted during cleaning of a connector (e.g., needleless access connector 30) with scrubbing cap 116, as explained in further detail below.

The second portion 150 of protrusion 146 includes an opening 162 formed therein at a proximal end 152 of protrusion 146 that provides access to a channel 164 formed within protrusion 146. The channel 164 may have a cylindrical cross-section shape and extend a majority of a length of the protrusion 146, such as to a location adjacent the base 128 of holder portion 124. The channel 164 is sized to receive therein the blunted cannula 88 of lock 86 on blood draw device 14. The blunted cannula 88 may form a press or interference fit with the channel 164, so as to aid in securing the lock 86 to scrubbing cap 116. In some embodiments, a proximal-most end of second portion 150 of protrusion 146 (and of channel 164 formed therein) is tapered outward to receive the blunted cannula 88 therein.

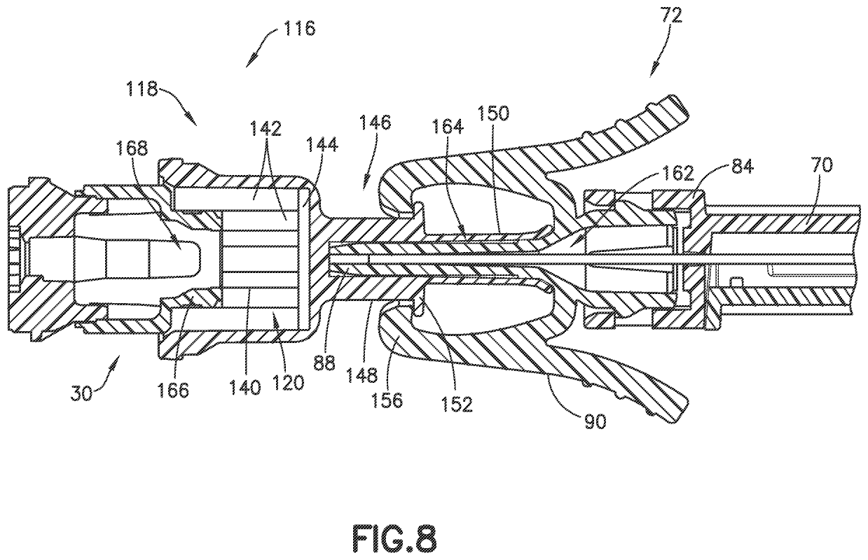

Following here below, use of the scrubbing cap 116 as a scrubbing device for cleaning and sterilizing of the proximal coupler 30 of catheter assembly 12 is described in accordance with one aspect of the disclosure. Use of the scrubbing cap 116 begins by removing the seal 122 from the scrubbing cap 116. After removal of the seal 122, the proximal coupler 30—which, as indicated above, may be configured as a female luer connection having a threaded outer connection 166 defining a tapered lumen or cavity 168—is inserted by the user into the foamed insert 120, as shown in FIG. 8. At this time, and as described above regarding a construction of insert 120, the fingers 142 of the insert 120 directly under the surfaces of the inserted portion of the proximal coupler 30 are depressed into the holder portion 124, while the fingers 142 located outside the periphery of the catheter remain upright and lay against the outer peripheral surface (i.e., threaded outer connection 166) of the proximal coupler 30 and the fingers 142 located directly under the lumen 168 of the proximal coupler 30 pass into the lumen 168. The separate reactions of the fingers 142 are facilitated by the fingers 142 being formed in a digitated manner to be separated from each other as described above.

Once the portion of the proximal coupler 30 has been inserted into the foamed insert 120 of the scrubbing cap 116, the scrubbing cap 116 is rotated relative to the proximal coupler 30. For example, the user may hold the proximal coupler 30 stationary while rotating the blood draw device 14. As the scrubbing cap 116 is locked in place relative to the blood draw device 14—via the connection of lock 86 to scrubbing cap 116 and the retaining of one of locking arms 90 within the retaining seat 160 formed on scrubbing cap 116—rotation of the blood draw device 14 causes a corresponding rotation of scrubbing cap 116. Rotation of the scrubbing cap 116 relative to the proximal coupler 30 may be done all in one direction or may be a back-and-forth twisting motion. The scrubbing cap 116 is rotated a sufficient number of times relative to the female luer connection of proximal coupler 30 to sufficiently kill any bacteria that the solution-impregnated foam insert 120 comes in contact with and/or to remove any biofilm from the outside peripheral surface and threaded outer connection 166 of proximal coupler 30 as well as the inside surface of the lumen 168 of the proximal coupler 30. In this way, both exterior surfaces and interior luminal surfaces of the proximal coupler 30 are scrubbed by the respective fingers 142 of the insert 120, causing the cleansing substance carried by the fingers 142 to disinfect the surfaces and remove any biofilm disposed thereon.

Upon completion of the cleaning and sterilizing of the proximal coupler 30 of catheter assembly 12 via scrubbing cap 116, the proximal coupler 30 is removed from the scrubbing cap 116. The scrubbing cap 116 may then be removed from blood draw device 14, with such removal performed by unclipping locking arms 90 from the lip 154 and retaining seat 160 of connector portion 126 and by withdrawing blunted cannula 88 from channel 164 of connector portion 126. After allowing the proximal coupler 30 to dry, the blood draw device 14 may then be connected to the catheter assembly 12 by coupling the lock 86 to proximal coupler 30—i.e., by inserting blunted cannula 88 into proximal coupler 30 and clipping locking arms 90 to proximal coupler 30.

While blood draw device 14 is shown and described in FIGS. 1-8 as including a coupling device 72 in the form of a lock 86 having a blunted cannula 88 and locking arms 90, it is recognized that coupling device 72 may be provide as any suitable connection or coupling that provides for engagement thereof with a proximal coupler 30 and for a distal end 104 of catheter 74 to pass through the coupling device 72 to access catheter assembly 12. It is also recognized that the specific construction of scrubbing cap 116 will be dependent on the configuration of coupling device 72, so as to provide for proper engagement and securing of the scrubbing cap 116 onto the coupling device 72. Described here below are a number of alternative constructions for coupling device 72 and scrubbing cap 116, according to additional aspects of the disclosure.

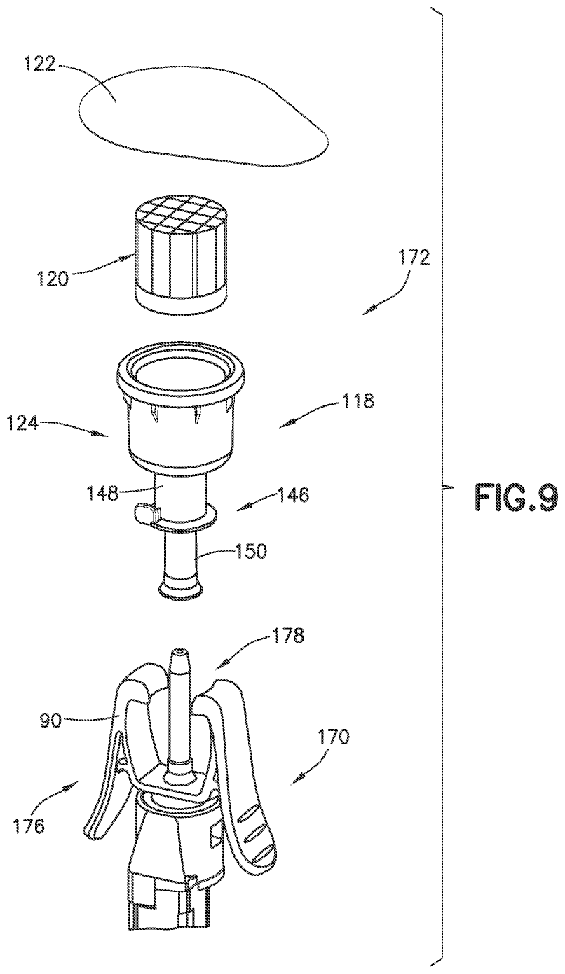

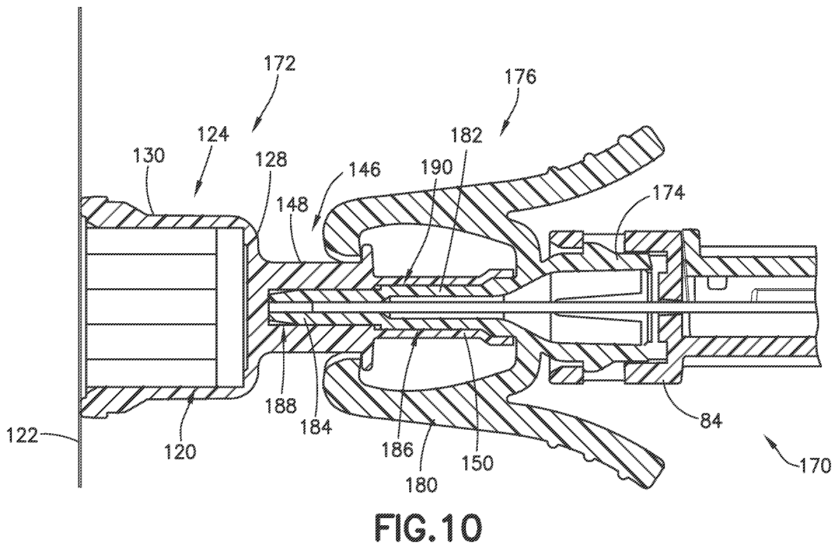

Referring first to FIGS. 9 and 10, a coupling device 170 and scrubbing cap 172 are shown in accordance with an embodiment of the disclosure. The coupling device 170 includes a proximal end configured as a threaded connection 174 that is configured to engage the coupler 84 of housing 70. The coupling device 170 is configured as a lock 176 that includes an elongated member 178 and locking arms 180 for coupling to a needleless access connector, with the elongated member 178 and locking arms 180 forming three points of contact therewith. In the present embodiment, the elongated member 178 has a hybrid construction—with elongated member 178 including a male luer portion 182 and a blunted cannula portion 184. The blunted cannula portion 184 extends out distally from male luer portion 182 and out past an end of locking arms 180, with the blunted cannula portion 184 having a smaller diameter as compared to male luer portion 182.

The scrubbing cap 172 is identical to scrubbing cap 116 previously shown and described in FIGS. 5-8 (and thus like numbers are used to identify identical components), except that the construction of a channel 186 formed in protrusion differs from the channel 164 previously described. That is, the channel 186 has a stepped configuration, with a first channel portion 188 formed within first portion 148 of protrusion 146 and a second channel portion 190 formed within second portion 150 of protrusion 146. The first channel portion 188 is formed to have a first channel diameter that is smaller than a second channel diameter of the second channel portion 190—with the first channel portion 188 thus sized to receive blunted cannula portion 184 of lock 176 and the second channel portion 190 thus sized to receive male luer portion 182. In some embodiments, a proximal-most end of second portion 150 of protrusion 146 (and of second channel portion 190 formed therein) is tapered outward to receive the male luer portion 182 of lock 176.

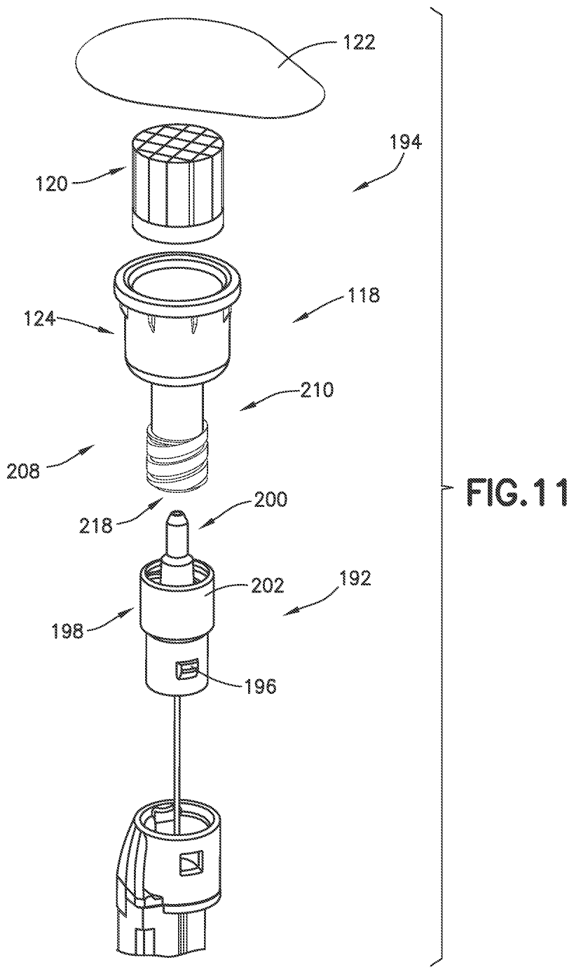

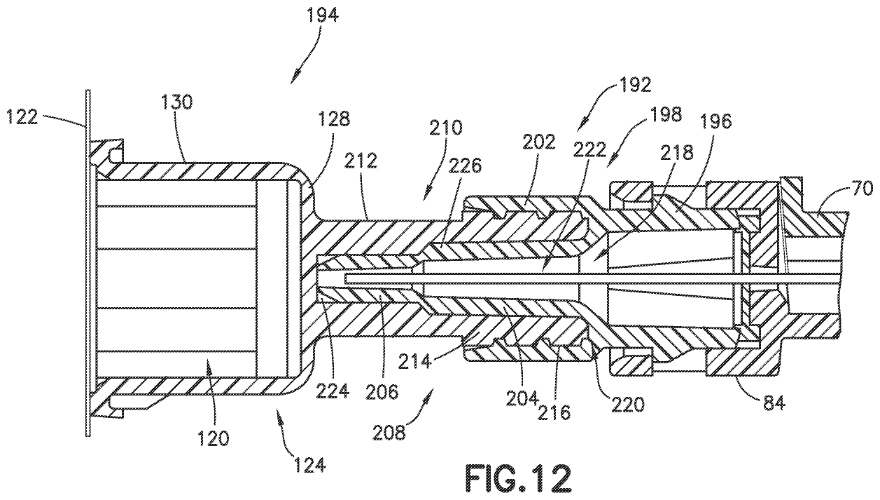

Referring next to FIGS. 11 and 12, a coupling device 192 and scrubbing cap 194 are shown in accordance with another embodiment of the disclosure. The coupling device 192 includes a proximal end configured as a threaded connection 196 that is configured to engage the coupler 84 of housing 70. The coupling device 192 is configured as a luer lock 198 that includes an elongated member 200 and an internally threaded collar 202 for coupling to a needleless access connector. In the present embodiment, the elongated member 200 has a hybrid construction—with elongated member 200 including a male luer portion 204 and a blunted cannula portion 206. The blunted cannula portion 206 extends out distally from male luer portion 204, with the blunted cannula portion 206 having a smaller diameter as compared to male luer portion 204.

The scrubbing cap 194 includes a holder portion 124 identical to scrubbing caps 116, 172 previously described, as well as a connector portion 208. The connector portion 208 is generally constructed as an elongated protrusion 210 extending outwardly from the base 128 of holder portion 124. The protrusion 210 may have a first portion 212 positioned adjacent the base 128 and a second portion 214 of the protrusion 210 positioned proximal from the first portion 212. The protrusion 210 may have a generally uniform outer diameter along a length thereof, except that second portion 214 is configured to have a threaded outer surface 216 that is configured to engage with the internally threaded collar 202 of luer lock 198, so as to secure scrubbing cap 194 to luer lock 198 of coupling device 192.

The second portion 214 of protrusion 210 includes an opening 218 formed therein at a proximal end 220 of protrusion 210 that provides access to a channel 222 formed within protrusion 210. The channel 222 may have a stepped configuration, with a first channel portion 224 formed within first portion 212 of protrusion 210 and a second channel portion 226 formed within second portion 214 of protrusion 210. The first channel portion 224 is formed to have a first channel diameter that is smaller than a second channel diameter of the second channel portion 226—with the first channel portion 224 thus sized to receive blunted cannula portion 206 of luer lock 198 and the second channel portion 226 thus sized to receive male luer portion 204.

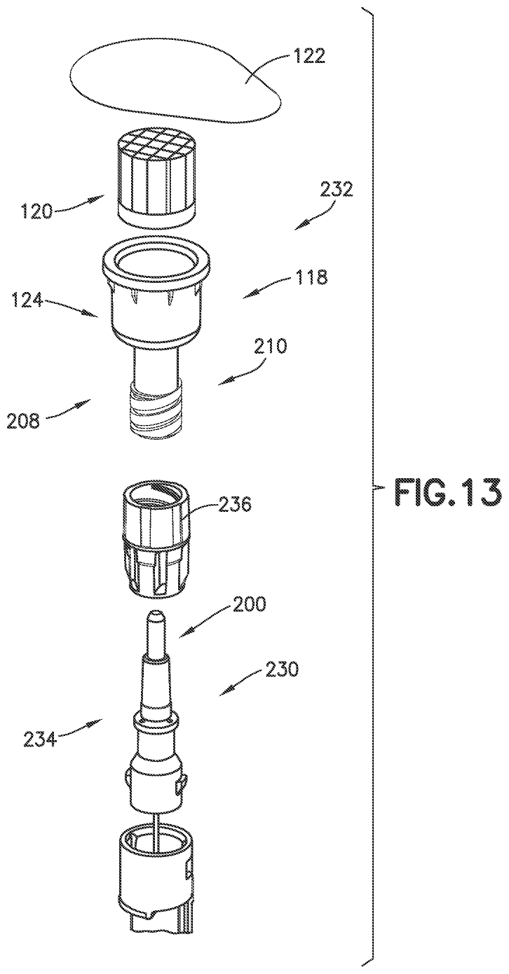

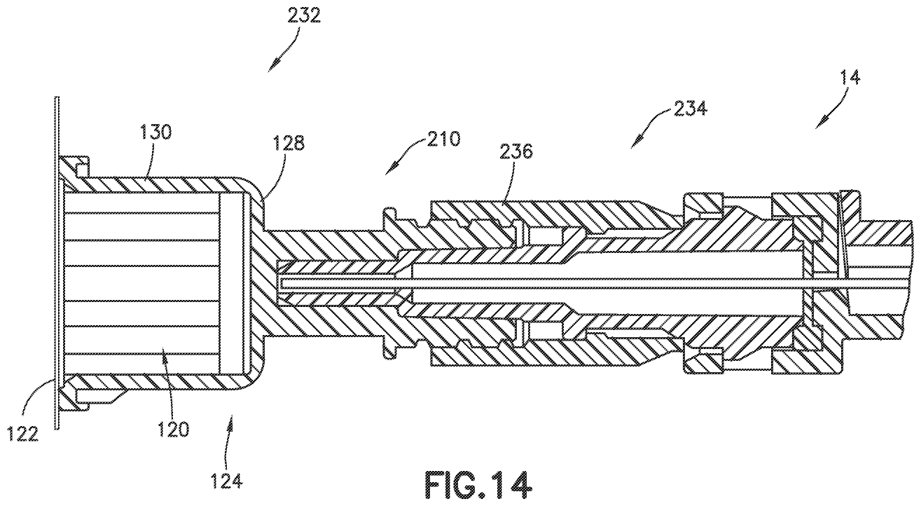

Referring next to FIGS. 13 and 14, a coupling device 230 and scrubbing cap 232 are shown in accordance with another embodiment of the disclosure. The scrubbing cap 232 is identical to the scrubbing cap 194 of FIGS. 11 and 12 and thus is not described in further detail here below. The coupling device 230 is substantially identical to the coupling device 192 of FIGS. 11 and 12, except that a luer lock 234 of the coupling device 230 includes an internally threaded rotating collar 236 that rotates relative to elongated member 200 and a remainder of the coupling device 230 (rather than collar 202 that is affixed to the luer lock 198 in coupling device 192) for coupling to a needleless access connector. The inclusion of rotating collar 236 allows for a remainder of the blood draw device 14 to remain stationary when the coupling device 230 is mated with the scrubbing cap 232 (or proximal coupler 30)—with only the rotating collar 236 being rotated to threadingly engage the scrubbing cap 232.

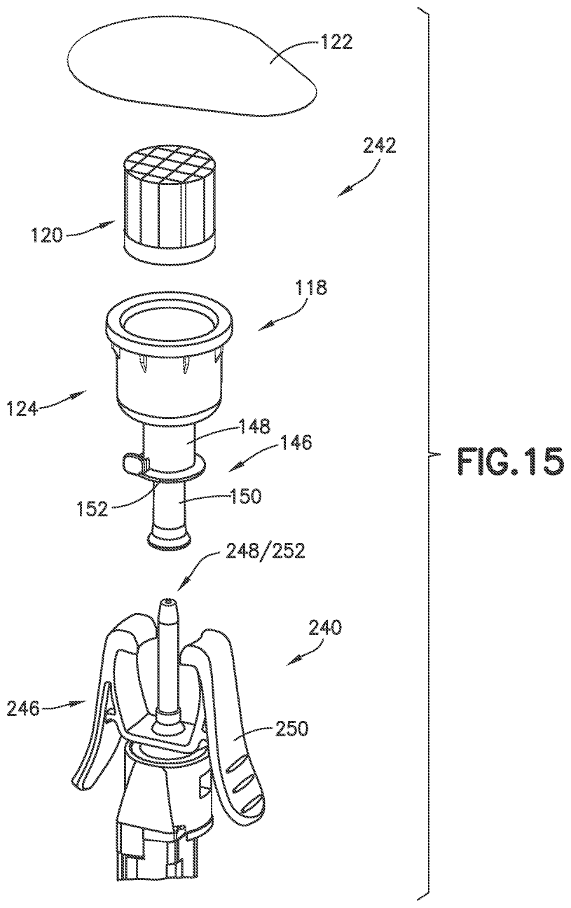

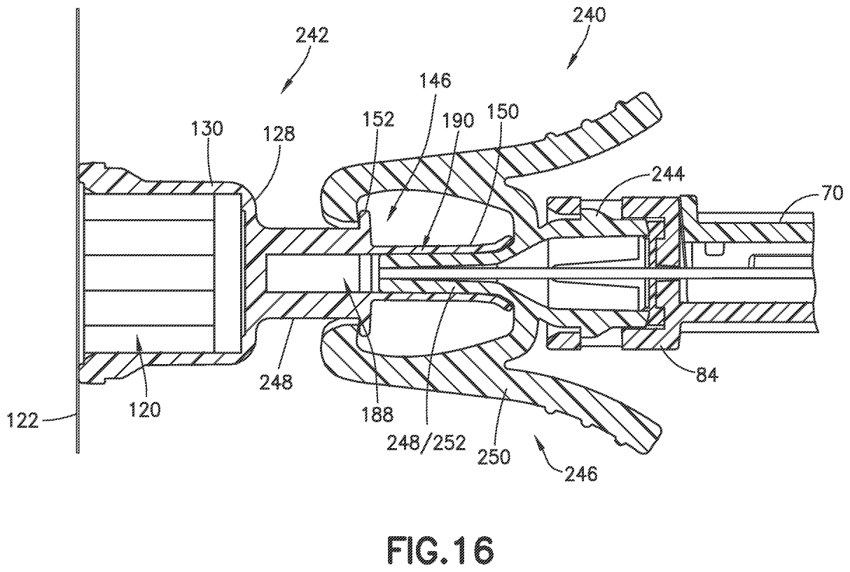

Referring next to FIGS. 15 and 16, a coupling device 240 and scrubbing cap 242 are shown in accordance with another embodiment of the disclosure. The coupling device 240 includes a proximal end configured as a threaded connection 244 that is configured to engage the coupler 84 of housing 70. The coupling device 240 is configured as a lock 246 that includes an elongated member 248 and locking arms 250 for coupling to a needleless access connector, with the elongated member 248 and locking arms 250 forming three points of contact therewith. In the present embodiment, the elongated member 248 is configured as a male luer 252.

The scrubbing cap 242 is identical to scrubbing cap 172 previously shown and described in FIGS. 9-10, expect that the scrubbing cap 242 mates in a different fashion with coupling device 240 (as compared to mating of scrubbing cap 172 with coupling device 174). That is, as the elongated member 248 of lock 246 of coupling device 240 includes only a male luer protrusion 252 (rather than having a hybrid construction with a male luer portion 182 and a blunted cannula portion 184, as with elongated member 178), only the second channel portion 190 formed within second portion 150 of protrusion 146 receives the elongated member 248 (i.e., male luer 252) of lock 246 therein, while the first channel portion 188 within first portion 148 of protrusion 146 remains open.

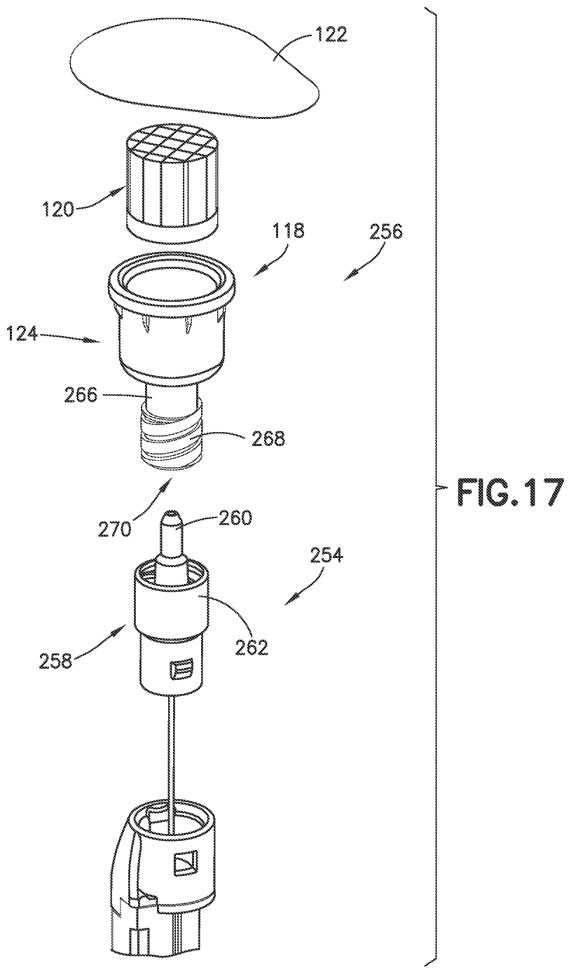

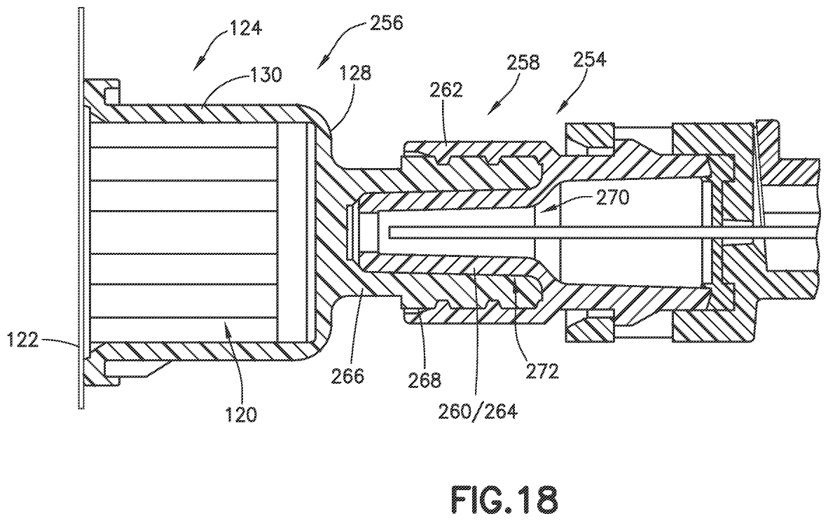

Referring next to FIGS. 17 and 18, a coupling device 254 and scrubbing cap 256 are shown in accordance with another embodiment of the disclosure. The coupling device 254 is similar to the coupling device 192 of FIGS. 11 and 12, in that coupling device 254 includes a luer lock 258 having an elongated member 260 and an internally threaded collar 262 for coupling to a needleless access connector. However, rather than elongated member 260 having a hybrid construction as does elongated member 200 (with a male luer portion 204 and a blunted cannula portion 206), elongated member 260 is instead constructed as a shortened male luer 264.

The scrubbing cap 256 includes a holder portion 124 identical to scrubbing caps previously described, as well as a connector portion 264. The connector portion 264 is generally constructed as shortened protrusion 266 extending outwardly from the base 128 of holder portion 124. The protrusion 266 may have a generally uniform outer diameter along a length thereof and is configured to have a threaded outer surface 268 that is configured to engage with the internally threaded collar 262 of luer lock 258, so as to secure scrubbing cap 256 to luer lock 258 of coupling device 254. The protrusion 266 includes an opening 270 formed therein at a proximal end of protrusion 210 that provides access to a channel 272 formed within protrusion 266. The channel 272 is formed to have a diameter that accommodates positioning therein of male luer 264.

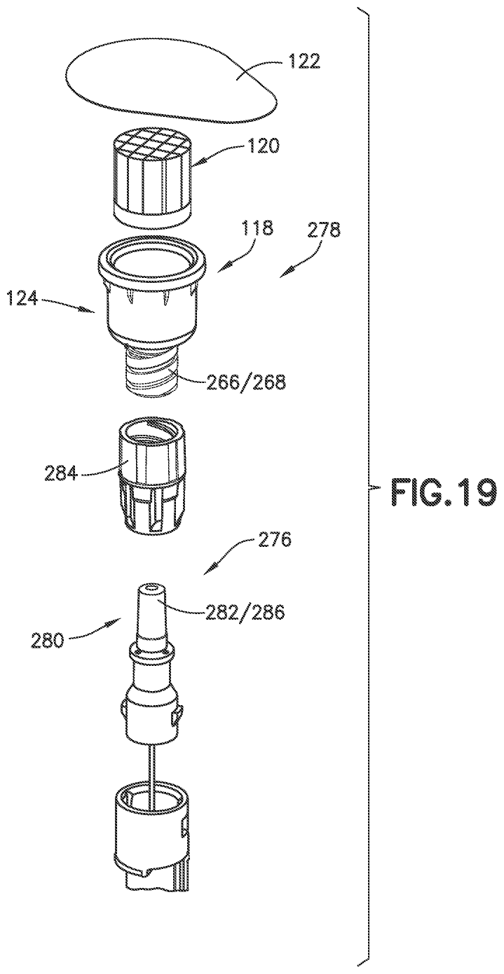

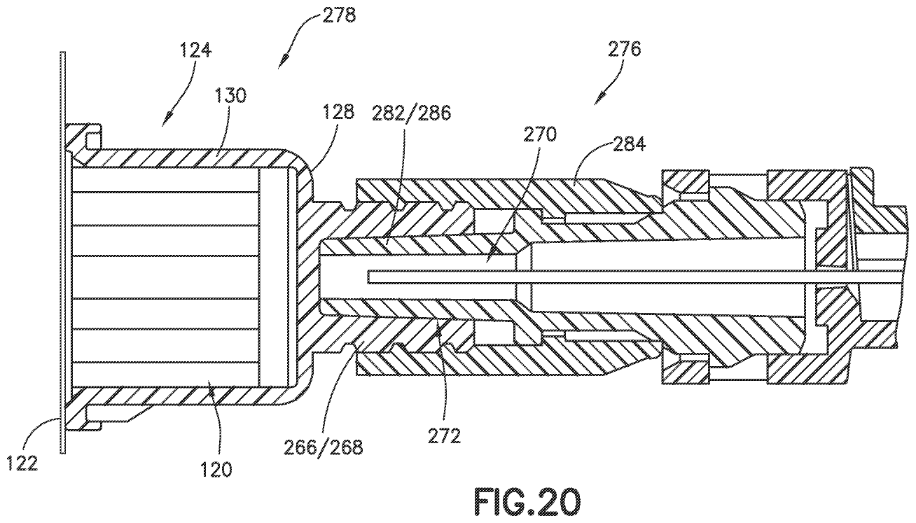

Referring next to FIGS. 19 and 20, a coupling device 276 and scrubbing cap 278 are shown in accordance with another embodiment of the disclosure. The scrubbing cap 278 is essentially identical to the scrubbing cap 256 of FIGS. 17 and 18 and thus is not described in further detail here below. The coupling device 276 is similar to the coupling device 230 of FIGS. 13 and 14, in that coupling device 276 includes a luer lock 280 having an elongated member 282 and an internally threaded rotating collar 284 that rotates relative to elongated member 282 and a remainder of the coupling device 276 for coupling to a needleless access connector. However, rather than elongated member 282 having a hybrid construction as does elongated member 200 (with a male luer portion 204 and a blunted cannula portion 206), elongated member 282 is instead constructed as a shortened male luer 286.

Beneficially, embodiments of the disclosure provide a scrubbing cap that is integrated with an instrument delivery device and that provides for disinfecting of a needleless connector or other access port of a peripheral IV catheter before connecting of the instrument delivery device thereto. The scrubbing cap is configured to securely mate with a coupling device positioned at the distal end of the instrument delivery device and is removable from the coupling device subsequent to disinfecting of the needleless connector/access port, so as to provide for a subsequent coupling of the instrument delivery device to the needleless connector/access port, to enable advancement of an instrument into the peripheral IV catheter.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An instrument delivery device coupleable to a catheter assembly including a catheter positionable intravenously in a patient, the instrument delivery device comprising:
an instrument configured to be inserted within the catheter;
a housing having a proximal end and a distal end and defining an inner volume configured to movably receive the instrument;
a coupling device positioned at the distal end of the housing, the coupling device comprising a lock configured to couple the housing to a proximal connector of the catheter assembly;
an advancement member configured to move relative to the housing to move the instrument between a first position, in which a distal end of the instrument is disposed within the housing or within the lock, and a second position, in which the distal end of the instrument is disposed within the catheter or past a distal tip of the catheter; and
a scrubbing cap engaged with the lock, the scrubbing cap comprising:
a scrubbing insert;
a housing including:
a connector portion configured to engage with the lock to secure the scrubbing cap to the instrument delivery device
a holder portion distal from the connector portion and including a cavity configured to retain the scrubbing insert therein; and
a seal attached to the holder portion to seal the scrubbing insert within the cavity;
wherein the scrubbing cap is engaged with the lock in a manner that provides for removal of the scrubbing cap from the instrument delivery device.

2. The instrument delivery device of claim 1, wherein the connector portion comprises a protrusion having a distal end joined with the connector portion and a proximal end having an opening formed therein, the opening providing access to a channel formed within the protrusion.

3. The instrument delivery device of claim 2, wherein the lock comprises:
an elongated member insertable within the channel of the protrusion; and
a pair of locking arms positioned on opposing sides of the elongated member and enagageable with the protrusion.

4. The instrument delivery device of claim 1, wherein the protrusion of the connector portion includes:
a first portion having a first outer diameter;
a second portion positioned proximally from the first portion and having a second outer diameter less than the first outer diameter, the second portion including the opening; and
an annular flange disposed between the first portion and the second portion and forming a lip;
wherein the annular flange is positioned such that a hooked end portion of each of the pair of locking arms engages the lip of the annular flange when the elongated member is inserted into the channel.

5. The instrument delivery device of claim 4, wherein the first portion includes a pair of retaining stops formed on an outer surface thereof and adjacent the annular flange, the pair of retaining stops arranged to form a retaining seat that receives the hooked end portion of a respective locking arm of the pair of locking arms, with the retaining seat preventing circumferential displacement of the locking arm.

6. The instrument delivery device of claim 5, wherein the channel comprises:
a first channel portion formed in the first portion of the protrusion; and
a second channel portion formed in the second portion of the protrusion;
wherein the first channel portion has a first channel diameter and the second channel portion has a second channel diameter greater than the first channel diameter.

7. The instrument delivery device of claim 6, wherein the elongated member comprises a male luer portion and a blunted cannula portion extending out distally from the male luer portion, with the blunted cannula portion sized to fit in the first channel portion and the male luer portion sized to fit in the second channel portion.

8. The instrument delivery device of claim 6, wherein the elongated member comprises a male luer connection sized to fit in the second channel portion, and wherein the first channel portion remains open when the elongated member is inserted into the channel.

9. The instrument delivery device of claim 2, wherein the lock comprises:
an elongated member insertable within the channel of the protrusion; and
a collar positioned about the elongated member and including a threaded inner surface.

10. The instrument delivery device of claim 9, wherein the protrusion of the connector portion comprises a threaded outer surface configured to engage with the threaded inner surface of the collar to secure the scrubbing cap to the lock.

11. The instrument delivery device of claim 10, wherein the elongated member comprises a male luer connection sized to fit in the channel.

12. The instrument delivery device of claim 10, wherein the protrusion of the connector portion includes:
a first portion; and
a second portion positioned proximally from the first portion, the second portion including the opening;
wherein the threaded outer surface is formed on the second portion.

13. The instrument delivery device of claim 12, wherein the channel comprise:
a first channel portion formed in the first portion of the protrusion; and
a second channel portion formed in the second portion of the protrusion;
wherein the first channel portion has a first channel diameter and the second channel portion has a second channel diameter greater than the first channel diameter.

14. The instrument delivery device of claim 13, wherein the elongated member comprises a male luer portion and a blunted cannula portion extending out distally from the male luer portion, with the blunted cannula portion sized to fit in the first channel portion and the male luer portion sized to fit in the second channel portion.

15. The instrument delivery device of claim 11, wherein the cavity in the holder portion and the scrubbing insert are sized and reconfigured to receive a female luer connection of the proximal connector of the catheter assembly.

16. A method of using the instrument delivery device of claim 1, the method comprising:
removing the seal from the holder portion;
positioning the proximal connector of the catheter assembly into the holder portion, such that the proximal connector contacts the scrubbing insert; and
cleaning the proximal connector with the scrubbing insert via application of a twisting motion to the instrument delivery device.

17. The method of claim 16, comprising:
detaching the scrubbing cap from the lock subsequent to cleaning of the proximal connector; and
coupling the lock to the proximal connector;
wherein, upon coupling of the lock to the proximal connector, the instrument delivery device is operable to move the instrument between from the first position to the second position.

18. A method of manufacturing an instrument delivery device coupleable to a catheter assembly including a catheter positionable intravenously in a patient, the method comprising:
providing an instrument delivery device comprising:
an instrument configured to be inserted within the catheter;
a housing having a proximal end and a distal end and defining an inner volume configured to movably receive the instrument;
a coupling device positioned at the distal end of the housing, the coupling device comprising a lock configured to couple the housing to a proximal connector of the catheter assembly; and
an advancement member configured to move relative to the housing to move the instrument between a first position, in which a distal end of the instrument is disposed within the housing or within the lock, and a second position, in which the distal end of the instrument is disposed within the catheter or past a distal tip of the catheter; and
engaging a scrubbing cap with the lock, the scrubbing cap comprising:
a scrubbing insert;
a housing including:
a connector portion configured to engage with the lock to secure the scrubbing cap to the instrument delivery device; and
a holder portion distal from the connector portion and including a cavity configured to retain the scrubbing insert therein; and
a seal attached to the holder portion to seal the scrubbing insert within the cavity;
wherein the scrubbing cap is engaged with the lock in a manner that provides for removal of the scrubbing cap from the instrument delivery device.

19. The method of claim 18, wherein engaging the scrubbing cap with the lock comprises:
inserting an elongated member of the lock into a channel formed in the connector portion of the scrubbing cap; and
pinching a pair of locking arms of the lock onto an annular flange formed about a protrusion of the connector portion.

20. The method of claim 19, wherein a locking arm of the pair of locking arms is secured between a pair of retaining stops formed on the protrusion of the connector portion, thereby preventing circumferential displacement of the pair of locking arms relative to the connector portion.
